(19) United States
(12) Patent Application Publication
(10) Pub. No.: US 2019/0240450 A1
(43) Pub. Date: Aug. 8, 2019

(54) COVER APPLICATION DEVICE AND MEDICAL ELONGATED BODY SET

(71) Applicant: TERUMO KABUSHIKI KAISHA, Tokyo (JP)

(72) Inventor: Tomoya OIWA, Fujinomiya-shi (JP)

(73) Assignee: TERUMO KABUSHIKI KAISHA, Tokyo (JP)

(21) Appl. No.: 16/151,611

(22) Filed: Oct. 4, 2018

(30) Foreign Application Priority Data
Feb. 2, 2018 (JP) 2018-016956

(51) Int. Cl. A61M 25/00; A61M 25/02

(52) U.S. Cl. CPC A61M 25/0069; A61M 25/02; A61M 2025/0681; A61M 2025/0293; A61M 2025/0246; A61M 2025/0079

(57) ABSTRACT

A cover application device makes it possible to easily impart desired slidability to a medical elongated body by putting a cover portion on a distal portion of a guide wire. The cover application device has the cover portion that is to be put on the distal portion of the guide wire, and an attachment tool having a lumen into which the guide wire is insertable and having an outer peripheral surface covered with the cover portion.

### CROSS-REFERENCES TO RELATED APPLICATIONS

This application claims the benefit of Japanese Application No. 2018-016956 filed on Feb. 2, 2018, the entire content of which is incorporated herein by reference.

### TECHNICAL FIELD

The present invention relates to a cover application device and a medical elongated body set.

### BACKGROUND

It is known to cover a surface of elongated medical devices, such as guide wires and catheters, with a highly slidable coating such as a hydrophilic coating for improving slidability inside a living body or other devices. One example of such a device is disclosed in JP-A-2010-259772. The slidability required for the coating varies depending on the geometry of the target vessel, such as the degree of bending and any stenosis, frictional resistance on an inner surface of the vessel, etc. Therefore, an operator must select a guide wire having slidability suitable for the application, so that the operator encounters little resistance in operating the guide wire, for example, inside a vessel or inside a catheter.

However, different guide wires can have various specifications for distal end stiffness and outer diameter. Therefore, it can be difficult to select a guide wire in which all of the specifications, including slidability, satisfy the particular application.

### SUMMARY

The present disclosure has been made in order to solve the above-described problem, and an object thereof is to provide a cover application device and a medical elongated body set, which make it possible to easily impart desired slidability to a medical elongated body.

According to the present disclosure, in order to achieve the above-described object, there is provided a cover application device for putting a cover portion on a distal portion of a medical elongated body. The cover application device has the cover portion that is to be put on the distal portion of the medical elongated body, and an attachment tool having a lumen into which the medical elongated body is insertable and having at least a portion of an outer peripheral surface covered with the cover portion.

According to the present disclosure, in order to achieve the above-described object, there is provided a medical elongated body set having the cover application device and the medical elongated body.

The cover application device configured as described above allows the cover portion to be put on the medical elongated body so that desired slidability can be easily imparted to the distal portion of the medical elongated body.

The cover portion may have a cover main body and an outer covering layer which covers at least a portion of an outer surface of the cover main body and is formed of a material different from that of the cover main body. In this manner, it is easy to optionally adjust the slidability of the outer surface of the cover portion.

The cover portion may have a proximal side opening portion which is open on a proximal side, and an attachment portion which is located on a distal side of the proximal side opening portion and to which a distal portion of the medical elongated body is to be abutted. In this manner, the distal portion of the medical elongated body presses the attachment portion. Accordingly, the cover portion can be separated from the attachment tool, and can be moved to the distal portion of the medical elongated body. In addition, the distal portion of the medical elongated body is abutted to the attachment portion. In this manner, a position of the cover portion with respect to the medical elongated body is defined. Therefore, the cover portion can be put on a proper position of the medical elongated body.

A thickness of a proximal portion of the cover portion may be equal to or smaller than a thickness of a distal portion of the cover portion. In this manner, if the cover portion is put on the distal portion of the medical elongated body, the proximal portion of the cover portion is less likely to protrude from the medical elongated body. Therefore, even if a force is applied to the cover portion from the proximal side, the force is less likely to be applied to the proximal portion of the cover portion. Therefore, when a medical device is delivered along the medical elongated body having the cover portion put thereon, the cover portion can be prevented from being turned over from the medical elongated body.

The attachment tool may have a communication section which allows the outer peripheral surface and the lumen to communicate with each other. In this manner, when the cover portion is put on the medical elongated body, the cover portion is likely to be detached from the attachment tool, thereby improving operability.

The communication section may be a slit which extends from a distal end to a proximal end of the outer peripheral surface. In this manner, the cover portion is likely to be detached from the attachment tool by changing a clearance of the slit, thereby improving the operability.

The communication section may be a hole portion. In this manner, water can be supplied to the outer peripheral surface from the lumen of the attachment tool via the hole portion, and friction is reduced between the outer peripheral surface of the attachment tool and the cover portion. Therefore, the cover portion is likely to be detached from the attachment tool, thereby improving the operability.

The friction coefficient of an inner surface of the cover portion may be higher than the friction coefficient of the outer covering layer. In this case, when the cover portion is put on the medical elongated body, the inner surface of the cover portion having the high friction coefficient comes into contact with the medical elongated body. Thus, the cover portion is less likely to be detached from the medical elongated body, thereby improving safety.

The cover portion may come into contact with an outer peripheral surface of the attachment tool, in a state where at least a portion of the outer covering layer is turned inside out so as to face inward. In this manner, when the cover portion is put on the medical elongated body, the outer covering layer comes into contact with the medical elongated body. In this manner, the outer covering layer of the cover portion comes into contact with the outer peripheral surface of the attachment tool. Accordingly, the cover portion can be easily detached from the attachment tool and easily put on the medical elongated body. In addition, until the cover portion is put on the medical elongated body, the outer covering layer does not come into contact with a portion other than the outer peripheral surface of the attachment tool. Therefore, the slidability of the outer covering layer is not changed, and the outer covering layer can be kept clean.

The outer covering layer may be formed of a hydrophilic polymer. Thus, when the cover portion is put on the medical elongated body, improved slidability can be imparted to the distal portion of the medical elongated body. In addition, in a case where the outer covering layer comes into contact with the outer peripheral surface of the attachment tool, the outer covering layer is likely to slide on the outer peripheral surface of the attachment tool. In this manner, the cover portion is likely to be detached from the attachment tool, thereby improving the operability.

According to the medical elongated body set configured as described above, the cover portion can be put on the medical elongated body by using the cover application device. Therefore, desired slidability can be easily imparted to the distal portion of the medical elongated body.

### BRIEF DESCRIPTION OF THE DRAWINGS

FIG. 1 is a plan view illustrating a medical elongated body set according to an embodiment.

FIG. 2 is an exploded cross-sectional view of a cover application device.

FIG. 3 is a cross-sectional view of the cover application device.

FIGS. 4(A) and 4(B) are cross-sectional views for describing an operation for causing the cover application device to provide a guide wire with a cover portion. FIG. 4(A) illustrates a state before the cover portion is provided, and FIG. 4(B) illustrates a state after the cover portion is provided.

FIGS. 5(A) and 5(B) are plan views illustrating modification examples of the cover application device. FIG. 5(A) illustrates a first modification example, and FIG. 5(B) illustrates a second modification example.

FIGS. 6(A)-6(C) are plan views illustrating modification examples of the cover application device. FIG. 6(A) illustrates a third modification example, FIG. 6(B) illustrates a fourth modification example, and FIG. 6(C) illustrates a fifth modification example.

FIGS. 7(A) and 7(B) are cross-sectional views illustrating a sixth modification example of the cover application device. FIG. 7(A) illustrates a state before the cover portion is provided, and FIG. 7(B) illustrates a state after the cover portion is provided.

### DETAILED DESCRIPTION

Hereinafter, an embodiment according to the present disclosure will be described with reference to the drawings. Dimensions in the drawings are exaggerated and different from actual dimension for convenience of description, in some cases. In addition, the same reference numerals will be given to configuration elements having substantially the same functional configuration, and thus, repeated description will be omitted. In the description herein, a side of a device which is inserted into a blood vessel will be referred to as a “distal side”, and an operating hand side will be referred to as a “proximal side”.

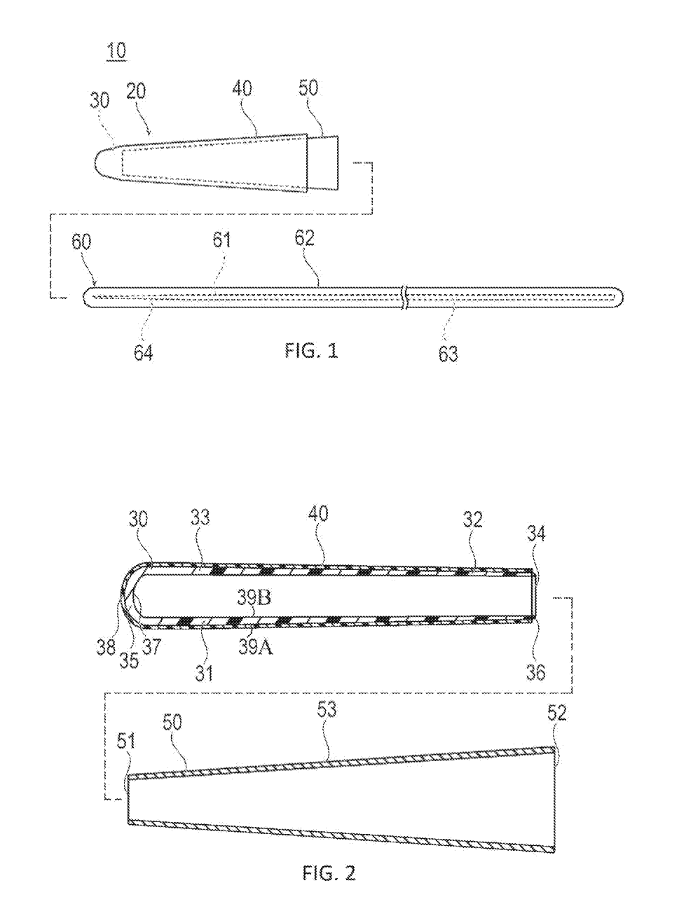

As illustrated in FIG. 1, a medical elongated body set 10 according to the present embodiment changes slidability of a distal portion of a guide wire 60 serving as a medical elongated body. The medical elongated body set 10 is configured to include the guide wire 60 and a cover application device 20.

The cover application device 20 is adapted to put a cover portion 30 on the distal portion of the guide wire 60 in order to change the slidability of the distal portion of the guide wire 60. The cover application device 20 includes the cover portion 30 and an attachment tool 50 for guiding the guide wire 60 into the cover portion 30. The cover portion 30 and the attachment tool 50 are used in a combined state.

As illustrated in FIG. 2, the cover portion 30 before being combined with the attachment tool 50 includes a cover main body 31 and an outer covering layer 40. The cover main body 31 has a cylindrical shape. The proximal side is open, and the distal side is closed. The cover portion 30 includes a cover proximal portion 32 located on the proximal side, and a cover distal portion 33 located on the distal side. The cover proximal portion 32 includes a proximal side opening portion 34 which is open on the proximal side. The cover distal portion 33 includes an attachment portion 35 which closes a lumen on the distal side. Furthermore, an outer edge portion 36 of the proximal side opening portion 34 of the cover portion 30 is chamfered in a curved surface or in a plane. The outer edge portion 36 may, alternatively, not be chamfered. An outer diameter and an inner diameter of the cover portion 30 decrease toward the proximal side. The inner diameter of the cover proximal portion 32 is smaller than the outer diameter of the guide wire 60. A thickness of the cover portion 30 decreases toward the proximal side. Therefore, although details will be described later, if the cover portion 30 covers the guide wire 60, the proximal portion of the cover portion 30 is in a stretched state. In this manner, the proximal portion of the cover portion 30 comes into close contact with a wire outer surface 65 which is an outer surface of the guide wire 60, by using a self-contractible force. Accordingly, the cover portion 30 is less likely to be detached from the guide wire 60, thereby improving safety. The outer diameter and the inner diameter of the cover portion 30 may be constant along an axial direction. In addition, the thickness of the cover portion 30 may decrease toward the distal side, or may be constant along the axial direction.

The attachment portion 35 is formed to have a substantially uniform thickness in the embodiment, but may also vary depending on sites. A proximal surface 37 of the attachment portion 35 located inside the cover distal portion 33 is a surface to which the guide wire 60 is to be abutted. The proximal surface 37 is formed in a concave shape having a curvature radius which substantially coincides with a curvature radius of the distal end of the guide wire 60. The proximal surface 37 of the attachment portion 35 comes into contact with the guide wire 60. In this manner, the cover portion 30 can be located at a proper position with respect to the guide wire 60. As long as the guide wire 60 can be abutted to the attachment portion 35, structure such as a through-hole having the inner diameter smaller than the inner diameter of the cover portion 30 may be formed in the attachment portion 35.

A distal surface 38 of the attachment portion 35 is formed in a convexly curved surface. The distal surface 38 is a site which is to be abutted to a biological tissue. Since the distal surface 38 is the curved surface, it is possible to prevent the biological tissue from being damaged. In a case where the distal surface 38 is covered by the outer covering layer 40, the distal surface 38 comes into contact with the biological tissue via the outer covering layer 40.

The outer covering layer 40 covers a substantial entirety of a cover outer surface 39A which is an outer surface of the cover main body 31. It is preferable that the outer covering layer 40 decreases frictional resistance, but the outer covering layer 40 may instead increase the frictional resistance. The outer covering layer 40 may include at least one type selected from the group consisting of hydrophilic coating, fluorine coating, and silicone coating. A material used for the hydrophilic coating may include a known hydrophilic substance formed of a cellulose-based polymer material, a polyethylene oxide-based polymeric material, an anhydrous maleic acid-based polymeric material (for example, a maleic anhydride copolymer such as a methyl vinyl ether-maleic anhydride copolymer), a acrylamide-based polymeric substance (for example, polyacrylamide, a block copolymer of glycidyl methacrylate-dimethylacrylamide (P(GMA-DMA))), water-soluble nylon, polyvinyl alcohol, and polyvinyl pyrrolidone. In addition, a material used for the fluorine coating may include at least one type selected from the group consisting of polytetrafluoroethylene (PTFE), tetrafluoroethylene-perfluoroalkyl vinyl ether copolymer (PFA), polychloro-trifluoroethylene (PCTFE), polyvinylidene fluoride (PVDF), polyvinylidene fluoride (PVF), tetrafluoroethylene-hexafluoropropylene copolymer (FEP), and tetrafluoroethylene-ethylene copolymer (PETFE). In addition, a material used for silicone coating may include silicone rubber and silicone resin.

Instead of being formed in the distal end of the cover main body 31, the outer covering layer 40 may be formed in the vicinity of the outer surface of the attachment portion 35. In this manner, without excessively decreasing the frictional resistance of the distal end of the guide wire 60, the blood vessel can be prevented from being perforated. Although a length is not particularly limited, the length in the axial direction of a range where the outer covering layer 40 of the cover portion 30 is not provided is 2 to 15 mm, for example. In addition, a layer formed of a material different from that of the outer covering layer 40 may be formed in the range where the outer covering layer 40 of the cover portion 30 is not provided. In addition, the outer covering layer 40 may be partially provided in, for example, a mesh shape, a spiral shape, or a dot shape. By partially providing the outer covering layer 40, anisotropy can be achieved in a sliding direction.

The outer covering layer 40 may be omitted. In this case, the cover outer surface 39A is exposed, thereby enabling the guide wire 60 to be provided with slidability.

A cover inner surface 39B which is the inner surface located in the lumen of the cover main body 31 is not provided with a slidability-improving covering or coating. The cover inner surface 39B has a friction coefficient higher than that of the outer covering layer 40. Therefore, when the cover portion 30 is put on the guide wire 60, the cover portion 30 comes into close contact with the guide wire 60. In this manner, it is possible to prevent the cover portion 30 from being deviated from the guide wire 60. The cover inner surface 39B may be covered or coated with a material having the high friction coefficient. The material having the high friction coefficient may include silicone rubber and silicone resin. In addition, the cover inner surface 39B may be provided with an uneven shape in order to raise the friction coefficient.

It is preferable that a configuration material of the cover main body 31 is a material which can expand and contract. The configuration material may include synthetic rubber such as latex rubber, isoprene rubber, styrene butadiene rubber, nitrile butadiene rubber, chloroprene rubber, polyurethane rubber, and silicone rubber, and synthetic rubber obtained in such a way that these materials are prevulcanized (in other words, aged) by using sulfur, peroxide, or radiation, or a thermoplastic elastomer including polyurethane, SBS, SEBS, SIS, and polyolefin.

It is preferable to set the length in the axial direction of the cover portion 30 so that the cover portion 30 is less likely to be detached from the guide wire 60. Although the length is not particularly limited, the length in the axial direction of the cover portion 30 is 30 to 500 mm, for example.

It is preferable that operability of the guide wire 60 is less affected by the thickness of the cover portion 30. Although the thickness is not particularly limited, the thickness of the cover portion 30 is 0.005 to 0.05 mm, for example.

The attachment tool 50 is a tubular body whose outer diameter and inner diameter decrease in a tapered shape toward the distal side. The attachment tool 50 includes a first opening portion 51 on the distal side, and a second opening portion 52 on the proximal side. An outer peripheral surface 53 of the attachment tool 50 is a surface covered by the cover portion 30. A minimum diameter of the attachment tool 50, that is, the inner diameter of the first opening portion 51 is larger than a maximum outer diameter of the guide wire 60 so that the guide wire 60 can pass through the first opening portion 51.

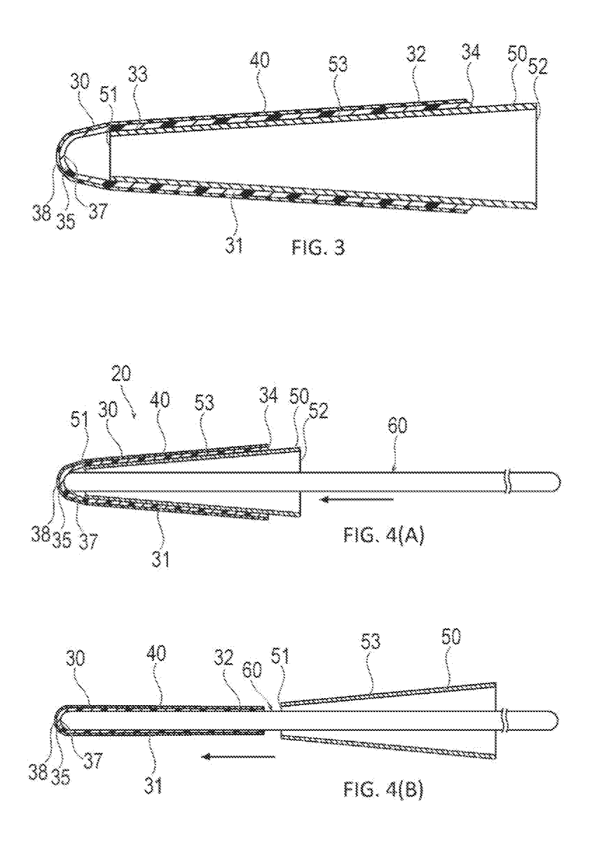

As illustrated in FIGS. 1 and 3, the cover portion 30 and the attachment tool 50 are combined with each other so as to configure the cover application device 20. In the cover application device 20, the distal portion of the attachment tool 50 is inserted from the proximal side opening portion 34 of the cover portion 30. The distal portion of the attachment tool 50 is close to the proximal surface 37 of the attachment portion 35. The distal portion of the attachment tool 50 may or may not come into contact with the proximal surface 37. The proximal portion of the cover portion 30 is stretched so that the inner diameter and the outer diameter are widened, and comes into close contact with the outer peripheral surface 53 of the attachment tool 50. At least a portion of the proximal portion of the attachment tool 50 is exposed without being covered by the cover portion 30. This exposed site is a site gripped and operated by an operator.

A configuration material of the attachment tool 50 is not particularly limited. However, the configuration material may include general purpose metal such as stainless steel and general purpose resins such as polyethylene, polypropylene, polystyrene, polyvinyl chloride, polyester, polymethacrylate, polyacrylate, polyoxymethylene, polyamide, and polyurethane, or a combination of two or more of these materials.

The guide wire 60 guides a medical device such as a catheter to a target position in a biological lumen such as a blood vessel. As illustrated in FIG. 1, the guide wire 60 includes an elongated core 61 and a covering material 62 for covering the core 61.

The core 61 includes a proximal shaft 63 located on the proximal side, and a distal shaft 64 located on the distal side of the proximal shaft 63. The proximal shaft 63 is a site extending from the proximal end toward the distal side of the core 61 and having a substantially constant outer diameter. The distal shaft 64 is a site whose outer diameter decreases in a tapered shape toward the distal side. The covering material 62 covers the core 61, and has a substantially constant outer diameter.

A configuration material of the core 61 is not particularly limited. However, the configuration material may include superelastic alloys such as Ni—Ti alloys and cobalt alloys, or stainless steel. A configuration material of the covering material 62 is not particularly limited. However, the configuration material may include a urethane resin. The wire outer surface 65 which is the outer surface of the covering material 62 may be provided with hydrophilic coating, fluorine coating, or silicone coating in order to have slidability.

A structure of the guide wire 60 is not limited to the above-described form. For example, in the distal portion of the guide wire 60, a coil may be located on the outer surface. In addition, the coil incorporated in a resin may be located in the distal portion of the guide wire 60. Alternatively, the distal portion of the guide wire 60 may be a tapered wire whose diameter decreases toward the distal side. Alternatively, the distal portion of the guide wire 60 may include the coil having multiple layers. Alternatively, the distal portion of the guide wire 60 may include a pipe in which a slit is formed in a spiral shape.

Next, an operation of the medical elongated body set 10 according to the present embodiment will be described.

As illustrated in FIG. 4(A), the distal portion of the guide wire 60 whose distal portion needs to be provided with desired slidability is inserted into the second opening portion 52 of the cover application device 20. In this case, it is preferable to grip a site on the proximal side of the outer peripheral surface 53 of the attachment tool 50. The inner diameter of the second opening portion 52 is widened from the distal side toward the proximal side. Therefore, the distal portion of the guide wire 60 can be easily inserted into the second opening portion 52. The guide wire 60 inserted into the second opening portion 52 is guided to the proximal side opening portion 34 of the cover portion 30 by the attachment tool 50 having a tapered shape. Then, the guide wire 60 enters the lumen of the cover portion 30 from the proximal side opening portion 34. The distal end of the guide wire 60 passes through the first opening portion 51 of the attachment tool 50, and is attached to the proximal surface 37 of the attachment portion 35. In this manner, a force is applied to the proximal surface 37 from the guide wire 60. If the attachment tool 50 is held with fingers and the guide wire 60 is further moved to the distal side, the cover inner surface 39B slides on the outer peripheral surface 53 of the attachment tool 50. In this manner, the cover portion 30 moves to the distal side with respect to the attachment tool 50, and the cover portion 30 covers the guide wire 60 as illustrated in FIG. 4(B). The inner diameter of the cover proximal portion 32 is smaller than the outer diameter of the guide wire 60. Accordingly, in a stretched state, the cover proximal portion 32 comes into contact with the guide wire 60. Therefore, the cover proximal portion 32 comes into close contact with the guide wire 60 by using a self-contractible force. In addition, the thickness of the cover portion 30 decreases toward the proximal side. Accordingly, the cover portion 30 hardly protrudes from the wire outer surface 65 in the vicinity of the proximal side opening portion 34. Furthermore, the outer edge portion 36 of the proximal side opening portion 34 of the cover portion 30 is chamfered in a curved surface or in a plane. Therefore, even if a force is applied to the cover portion 30 from the proximal side, the force is less likely to be applied to the proximal portion of the cover portion 30. Therefore, when the medical device such as the catheter is delivered along the guide wire 60 having the cover portion 30 put thereon, the cover portion 30 can be prevented from being turned over from the guide wire 60.

After the cover portion 30 is completely moved from the attachment tool 50 to the guide wire 60, the guide wire 60 is moved to the distal side or the proximal side with respect to the attachment tool 50. In this manner, the guide wire 60 is pulled out from the attachment tool 50, and operation for putting the cover portion 30 on the distal portion of the guide wire 60 is completely carried out. Furthermore, the cover portion 30 is stretchable, and so the cover portion 30 may be detachable from the guide wire 60 by pulling and deforming the cover portion 30 with the fingers. In addition, the cover portion 30 may be broken so as to be detachable from the guide wire 60.

As described above, according to the present embodiment, there is provided the cover application device 20 for putting the cover portion 30 on the distal portion of the guide wire 60 (medical elongated body). The cover application device 20 has the cover portion 30 that is to be put on the distal portion of the guide wire 60, and the attachment tool 50 having the lumen into which the guide wire 60 is insertable and having the outer peripheral surface 53 covered with the cover portion 30.

The cover application device 20 configured as described above allows the cover portion 30 to be put on the guide wire 60 so that desired slidability can be easily imparted to the distal portion of the guide wire 60.

In addition, the cover portion 30 has the cover main body 31 and the outer covering layer 40 which covers at least a portion of the cover outer surface 39A of the cover main body 31 and is formed of a material different from that of the cover main body 31. In this manner, it is easy to adjust the slidability of the outer surface of the cover portion 30 to the desired slidability.

Furthermore, the cover portion 30 has the proximal side opening portion 34 which is open on the proximal side, and the attachment portion 35 which is located on the distal side of the proximal side opening portion 34 and to which the distal portion of the guide wire 60 is to be abutted. In this manner, the distal portion of the guide wire 60 presses the attachment portion 35. Accordingly, the cover portion 30 can be separated from the attachment tool 50, and can be moved to the distal portion of the guide wire 60. In addition, the distal portion of the guide wire 60 is abutted to the attachment portion 35. In this manner, a position of the cover portion 30 with respect to the guide wire 60 is defined. Therefore, the cover portion 30 can be put on a proper position of the guide wire 60.

In addition, the thickness of the proximal portion of the cover portion 30 is equal to or smaller than the thickness of the distal portion of the cover portion 30. In this manner, if the cover portion 30 is put on the distal portion of the guide wire 60, the proximal portion of the cover portion 30 is less likely to protrude from the wire outer surface 65. Therefore, for example, even if a force is applied to the cover portion 30 from the proximal side, the force is less likely to be applied to the proximal portion of the cover portion 30. Therefore, when the medical device such as the catheter is delivered along the guide wire 60 having the cover portion 30 put thereon, the cover portion 30 can be prevented from being turned over from the guide wire 60.

Furthermore, the friction coefficient of the cover inner surface 39B of the cover portion 30 is higher than the friction coefficient of the outer covering layer 40. In this manner, when the cover portion 30 is put on the guide wire 60, the cover inner surface 39B having the high friction coefficient comes into contact with the guide wire 60, and so the cover portion 30 is less likely to be detached from the guide wire 60, thereby improving safety.

In addition, the outer covering layer 40 may be formed of a hydrophilic polymer. Thus, when the cover portion 30 is put on the guide wire 60, improved slidability can be imparted to the distal portion of the guide wire 60.

The present invention is not limited only to the above-described embodiment, and various modifications can be made by those skilled in the art within the technical idea of the present invention. For example, the biological lumen into which the guide wire 60 is inserted is not limited to the blood vessel, and the biological lumen may be, for example, another type of vessel, a ureter, a bile duct, an oviduct, or a hepatic duct.

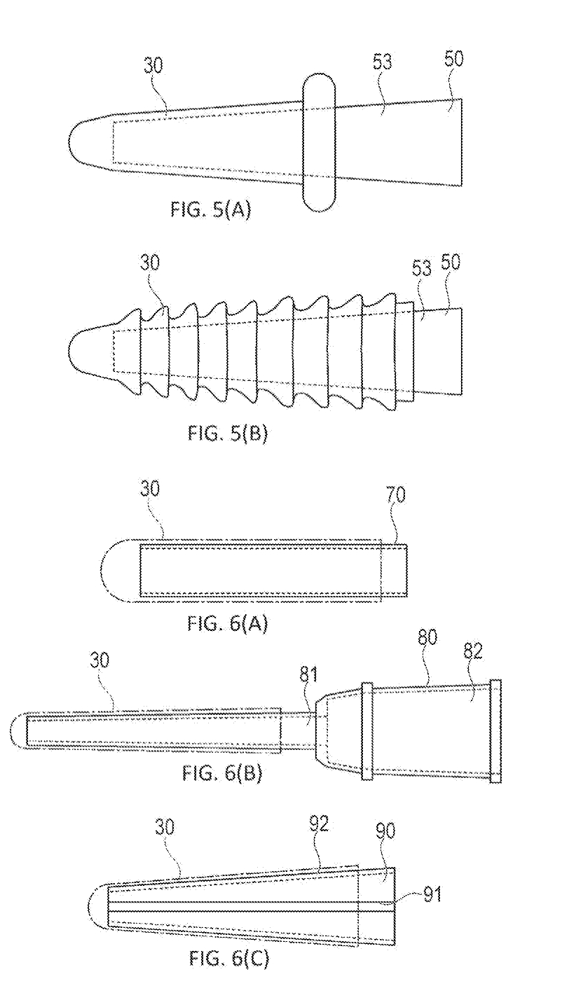

In addition, as in a first modification example illustrated in FIG. 5(A), a portion of the proximal side of the cover portion 30 covering the attachment tool 50 may be wound in a roll shape. After the wound cover portion 30 is moved to the guide wire 60, the cover portion 30 is stretched so that the wound portion is unwound. In this manner, a site of the proximal side of the cover portion 30 can be brought into close contact with the guide wire 60. The cover portion 30 moves to the guide wire 60 in a state where the cover portion 30 is wound so that the length in the axial direction is shortened, thereby improving workability.

Furthermore, as in a second modification example illustrated in FIG. 5(B), the cover portion 30 covering the attachment tool 50 may be repeatedly folded in a bellows shape on the outer peripheral surface 53 of the attachment tool 50. In this manner, the cover portion 30 is located in the attachment tool 50 in a state where the cover portion 30 is folded and the length in the axial direction is shortened. Therefore, the length in the axial direction of the attachment tool 50 can be shortened, thereby improving the workability.

In addition, as in a third modification example illustrated in FIG. 6(A), the inner diameter and the outer diameter of an attachment tool 70 may be substantially constant along the axial direction.

Furthermore, as in a fourth modification example illustrated in FIG. 6(B), an attachment tool 80 may include a hub 82 having a lumen for communicating with a cylindrical portion 81, on the proximal side of the cylindrical portion 81. The attachment tool 80 includes the hub 82 so as to be easily gripped, thereby improving the operability.

In addition, as in a fifth modification example illustrated in FIG. 6(C), an attachment tool 90 may have a slit 91 (communication section) extending from the distal end to the proximal end of an outer peripheral surface 92. The slit 91 allows the outer peripheral surface 92 of the attachment tool 90 and a lumen to communicate with each other. In this manner, when the cover portion 30 is put on the guide wire 60, the attachment tool 90 can be gripped so as to narrow a clearance of the slit 91, and so the cover portion 30 is likely to be detached from the attachment tool 90, thereby improving the operability.

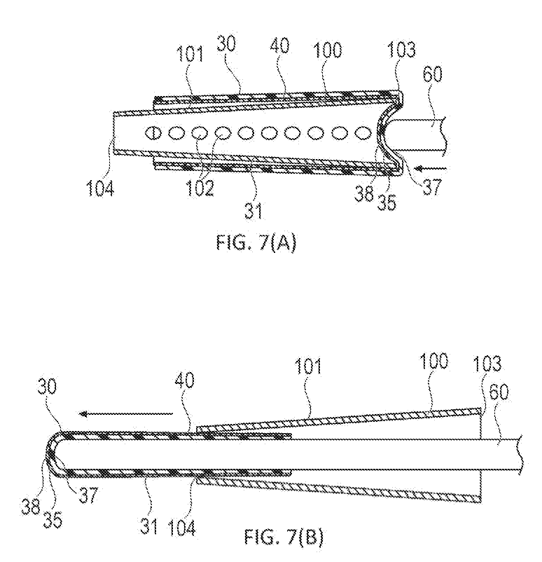

Furthermore, as in a sixth modification example illustrated in FIG. 7(A), an attachment tool 100 may have a hole portion 102 (communication section) which allows an outer peripheral surface 101 and a lumen to communicate with each other. The attachment tool 100 includes a first opening portion 104 on the distal side, and a second opening portion 103 on the proximal side. The cover portion 30 comes into contact with the outer peripheral surface 101 of the attachment tool 100, in a state where at least a portion of the outer covering layer 40 is turned inside out so as to face inward. The attachment portion 35 is located inside the second opening portion 103. The distal surface 38 of the attachment portion 35 enters the inside of the second opening portion 103. The outer covering layer 40 can come into contact with the outer peripheral surface 101. The outer covering layer 40 may be in close contact with the outer peripheral surface 101 without any clearance therebetween. In this state, if the attachment tool 100 is immersed in a saline solution, the saline solution flows from the first opening portion 104 through the hole portion 102, and is supplied between the outer covering layer 40 and the outer peripheral surface 101. For example, in a case of the hydrophilic coating, the outer covering layer 40 immersed in the saline solution shows improved slidability. Therefore, if the distal end of the guide wire 60 is attached to the proximal surface 37 of the attachment portion 35 and the guide wire 60 is moved to the distal side, the outer covering layer 40 smoothly slides on the outer peripheral surface 101. In this manner, as illustrated in FIG. 7(B), the cover portion 30 is put on the distal portion of the guide wire 60 while the cover portion 30 is restored so that the outer covering layer 40 faces outward. According to the sixth modification example, the outer covering layer 40 of the covering portion 30 in a state where the covering portion 30 is attached to the attachment tool 100 has slidability. Accordingly, the cover portion 30 can be easily detached from the attachment tool 100 and easily put on the guide wire 60. In addition, until the cover portion 30 is put on the guide wire 60, the outer covering layer 40 does not come into contact with a portion other than the outer peripheral surface 101 of the attachment tool 100. Therefore, the slidability of the outer covering layer 40 is not degraded (changed), and the outer covering layer 40 can be kept clean.

The detailed description above describes a cover application device and medical elongated body set disclosed by way of example. The invention is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. A cover application device for putting a cover portion on a distal portion of a medical elongated body, the device comprising:
a cover portion configured to be put on a distal portion of a medical elongated body; and
an attachment tool having a lumen into which the medical elongated body is insertable and having at least a portion of an outer peripheral surface covered with the cover portion.

2. The cover application device according to claim 1,
wherein the cover portion has
a cover main body, and
an outer covering layer which covers at least a portion of an outer surface of the cover main body and is formed of a material different from that of the cover main body.

3. The cover application device according to claim 1,
wherein the cover portion has
a proximal side opening portion which is open on a proximal side, and
an attachment portion which is located on a distal side of the proximal side opening portion and to which the distal portion of the medical elongated body is to be abutted.

4. The cover application device according to claim 1,
wherein a thickness of a proximal portion of the cover portion is equal to or smaller than a thickness of a distal portion of the cover portion.

5. The cover application device according to claim 1,
wherein the attachment tool has a communication section which allows the outer peripheral surface and the lumen to communicate with each other.

6. The cover application device according to claim 5,
wherein the communication section is a slit which extends from a distal end to a proximal end of the outer peripheral surface.

7. The cover application device according to claim 5,
wherein the communication section is a hole portion.

8. The cover application device according to claim 1,
wherein a friction coefficient of an inner surface of the cover portion is higher than a friction coefficient of the outer covering layer.

9. The cover application device according to claim 1,
wherein the cover portion comes into contact with an outer peripheral surface of the attachment tool in a state where at least a portion of the outer covering layer is turned inside out so as to face inward.

10. The cover application device according claim 1,
wherein the outer covering layer is formed of a hydrophilic polymer.

11. A medical elongated body set comprising:
a medical elongated body;
a cover portion configured to be put on a distal portion of a medical elongated body; and
an attachment tool having a lumen into which the medical elongated body is insertable and having at least a portion of an outer peripheral surface covered with the cover portion.

12. The medical elongated body set according to claim 11,
wherein the cover portion has
a cover main body, and
an outer covering layer which covers at least a portion of an outer surface of the cover main body and is formed of a material different from that of the cover main body.

13. The medical elongated body set according to claim 11,
wherein the cover portion has
a proximal side opening portion which is open on a proximal side, and
an attachment portion which is located on a distal side of the proximal side opening portion and to which the distal portion of the medical elongated body is to be abutted.

14. The medical elongated body set according to claim 11,
wherein a thickness of a proximal portion of the cover portion is equal to or smaller than a thickness of a distal portion of the cover portion.

15. The medical elongated body set according to claim 11,
wherein the attachment tool has a communication section which allows the outer peripheral surface and the lumen to communicate with each other.

16. The medical elongated body set according to claim 15,
wherein the communication section is a slit which extends from a distal end to a proximal end of the outer peripheral surface.

17. The medical elongated body set according to claim 15,
wherein the communication section is a hole portion.

18. The medical elongated body set according to claim 11,
wherein a friction coefficient of an inner surface of the cover portion is higher than a friction coefficient of the outer covering layer.

19. The medical elongated body set according to claim 11,
wherein the cover portion comes into contact with an outer peripheral surface of the attachment tool in a state where at least a portion of the outer covering layer is turned inside out so as to face inward.

20. The medical elongated body set according claim 11,
wherein the outer covering layer is formed of a hydrophilic polymer.
